(19) United States
(12) Patent Application Publication
(10) Pub. No.: US 2017/0145001 A1
(43) Pub. Date: May 25, 2017

(54) PROCESSES FOR PREPARING BREXPIPRAZOLE

(71) Applicant: SCINOPHARM TAIWAN, LTD., Tainan (TW)

(72) Inventors: Chia-Ying LEE, Tainan (TW); Shu Ting HUANG, Tainan (TW); Hsin-Chi WANG, Tainan (TW); Yuan-Xiu LIAO, Tainan (TW); Jiunn-Cheh GUO, Tainan (TW); Lung-Huang KUO, Tainan (TW)

(21) Appl. No.: 15/340,834

(22) Filed: Nov. 1, 2016

Related U.S. Application Data
(60) Provisional application No. 62/250,248, filed on Nov. 3, 2015.

(51) Int. Cl. C07D 409/12

(52) U.S. Cl. CPC C07D 409/12

(57) ABSTRACT

The present disclosure provides processes for preparing brexpiprazole. The present disclosure also provides processes for the purification of brexpiprazole. The processes for preparing and purifying brexpiprazole of the present invention provide substantial improvements over currently known methods. In certain embodiments, the conversion of Formula XI and XII to form XIII provides increased selectivity over previously reported methods. This offers increased yield and purity. The improved process for purifying brexpiprazole disclosed herein provides brexpiprazole with superior purity and is also more suitable for industrial production.

### CROSS-REFERENCES TO RELATED APPLICATIONS

This application claims priority to U.S. Provisional Application No. 62/250,248 filed on Nov. 3, 2015, the contents of which is herein incorporated by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Brexpiprazole is used for the treatment of schizophrenia and major depressive disorder. Brexpiprazole has been prepared by a process including reaction of 1-(benzo[b]-thiophen-4-yl)piperazine (II) with 7-(4-chlorobutoxy)quinolin-2(1H)-one (III). See, FIG. 1. Brexpiprazole has also been prepared by a process including reaction of 1-(benzo[b]thiophen-4-yl)piperazine (II) with 7-(4-chlorobutoxy)-3,4-dihydroquinolin-2(1H)-one (IV). See, FIG. 2. Known procedures for synthesis of brexpiprazole involve multiple steps using materials that are not commercially available. New processes for preparing brexpiprazole are needed. The present invention meets this need.

### BRIEF SUMMARY OF THE INVENTION

In one aspect, the present disclosure provides processes for preparing brexpiprazole.

In one embodiment, a process is provided comprising:
(1) contacting a compound of Formula XIa

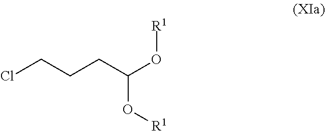

with a compound of Formula XII

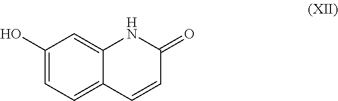 [SMILES: O=c1ccc2ccc(O)cc2[nH]1]

and a base in a first organic solvent to form a compound of Formula XIIIa;

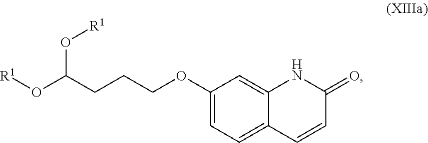

wherein each R¹ is independently C₁₋₈ alkyl, or optionally, both R¹ moieties in combination with the oxygen atoms to which each are attached form a 5-8 membered heterocycloalkyl ring;
(2) contacting a compound of Formula XIIIa
   with an acid in a second organic solvent to form a compound of Formula XIV

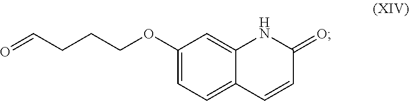 [SMILES: O=CCCCOc1ccc2ccc(=O)[nH]c2c1]

and
(3) contacting a compound of Formula XIV
   with a compound of Formula XV

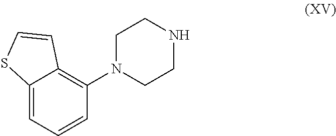 [SMILES: c1cc(N2CCNCC2)c2ccsc2c1]

or a salt thereof and a reducing agent in an organic solvent to form brexpiprazole.

In another embodiment, the process includes converting a compound of Formula VI

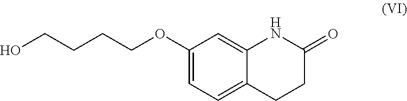 [SMILES: O=C1CCc2ccc(OCCCCO)cc2N1]

to brexpiprazole.

In some embodiments, converting the compound of Formula VI to brexpiprazole includes:
a) converting the compound of Formula VI to a compound of Formula VII

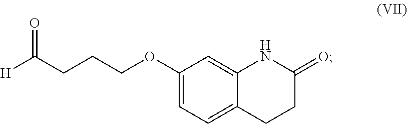 [SMILES: O=CCCCOc1ccc2c(c1)NC(=O)CC2]

b) contacting the compound of Formula VII with a compound of Formula II

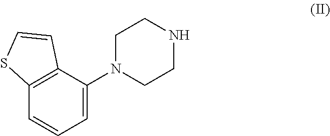 [SMILES: c1cc(N2CCNCC2)c2ccsc2c1]

to form a compound of Formula V
and

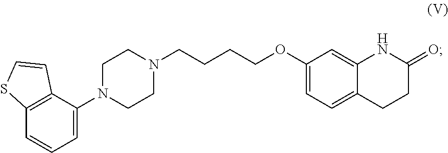 [SMILES: O=C1CCc2ccc(OCCCCN3CCN(c4cccc5sccc45)CC3)cc2N1]

c) converting the compound of Formula V to brexpiprazole.

Alternatively, converting the compound of Formula VI to brexpiprazole comprises:
a) converting the compound of Formula VI to a compound of Formula VII

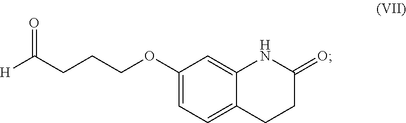 [SMILES: O=CCCCOc1ccc2c(c1)NC(=O)CC2]

b) converting the compound of Formula VII to a compound of Formula IX
   and

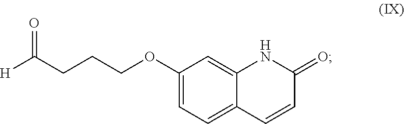 [SMILES: O=CCCCOc1ccc2ccc(=O)[nH]c2c1]

c) contacting the compound of Formula IX with a compound of Formula II

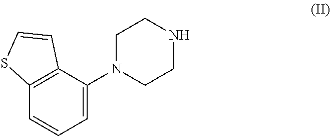 [SMILES: c1cc(N2CCNCC2)c2ccsc2c1]

to form brexpiprazole.

In some embodiments, converting the compound of Formula VI to brexpiprazole comprises:
i) converting the compound of Formula VI to a compound of Formula Villa

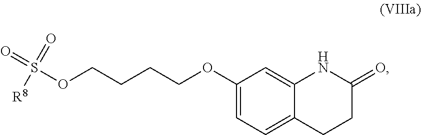

wherein R⁸ is selected from C₁₋₆ alkyl, C₁₋₆ haloalkyl, and C₆₋₁₀ aryl, wherein C₆₋₁₀ aryl is optionally substituted with one or more substituents independently selected from C₁₋₆ alkyl, halo, and nitro;
ii) contacting the compound of Formula VIIIa with a compound of Formula

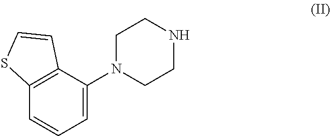 [SMILES: c1cc(N2CCNCC2)c2ccsc2c1]

to form a compound of Formula V
and

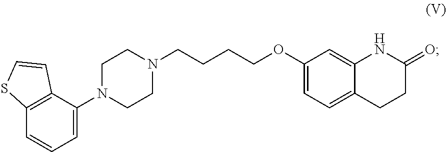 [SMILES: O=C1CCc2ccc(OCCCCN3CCN(c4cccc5sccc45)CC3)cc2N1]

iii) converting the compound of Formula V to brexpiprazole.

Alternatively, converting the compound of Formula VI to brexpiprazole includes:
a) converting the compound of Formula VI to a compound of Formula Villa

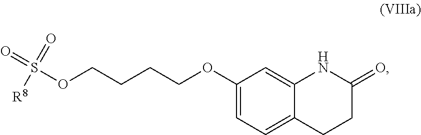

wherein R⁸ is selected from C₁₋₆ alkyl, C₁₋₆ haloalkyl, and C₆₋₁₀ aryl, wherein C₆₋₁₀ aryl is optionally substituted with one or more substituents independently selected from C₁₋₆ alkyl, halo, and nitro;
b) converting the compound of Formula Villa to a compound of Formula Xa
   and

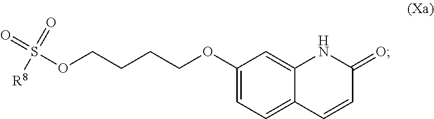

c) contacting the compound of Formula Xa with a compound of Formula II

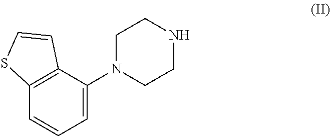 [SMILES: c1cc(N2CCNCC2)c2ccsc2c1]

to form brexpiprazole.

In some embodiments, the compound of Formula Villa is a compound of Formula VIII:

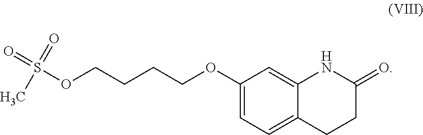 [SMILES: CS(=O)(=O)OCCCCOc1ccc2c(c1)NC(=O)CC2]

In another aspect, the disclosure provides processes for purifying brexpiprazole. In some embodiments, the process for purifying brexpiprazole is coupled with the process for preparing brexpiprazole.

In yet another aspect, the disclosure provides brexpiprazole prepared according to the processes described herein.

In still another aspect, the disclosure provides a pharmaceutical composition containing brexpiprazole prepared according to the processes described herein and a pharmaceutically acceptable excipient.

### BRIEF DESCRIPTION OF THE DRAWINGS

FIG. 1A shows a process for preparing brexpiprazole as disclosed in U.S. Pat. No. 7,888,362.

FIG. 1B shows a process for preparing brexpiprazole as disclosed in CN104844585A.

FIG. 2 shows a process for preparing brexpiprazole as disclosed in CN104829602A.

FIG. 3 shows a process for preparing brexpiprazole according to an embodiment of the present disclosure.

FIG. 4 shows a process for preparing brexpiprazole according to an embodiment of the present disclosure.

FIG. 5 shows a process for preparing brexpiprazole according to an embodiments of the present disclosure.

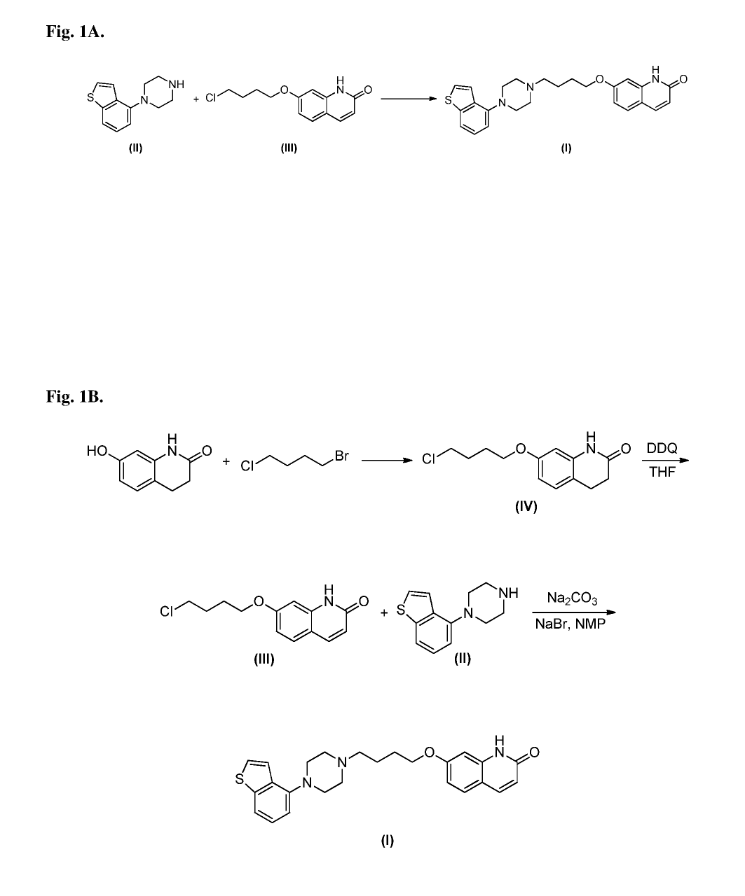

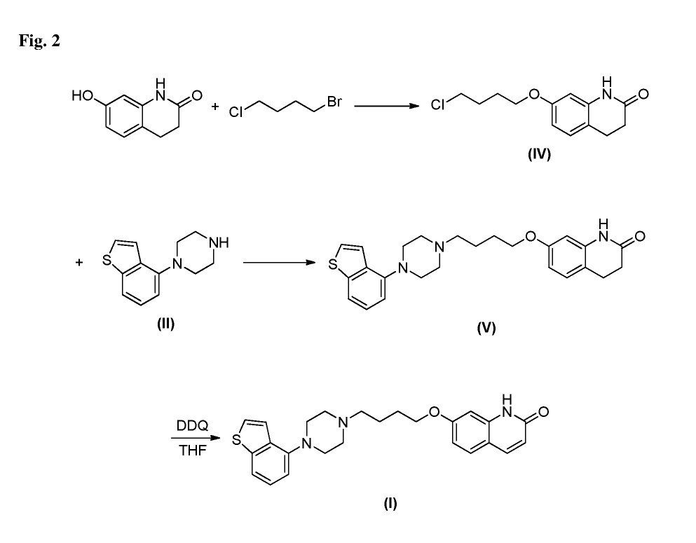

### DETAILED DESCRIPTION OF THE INVENTION

### I. General

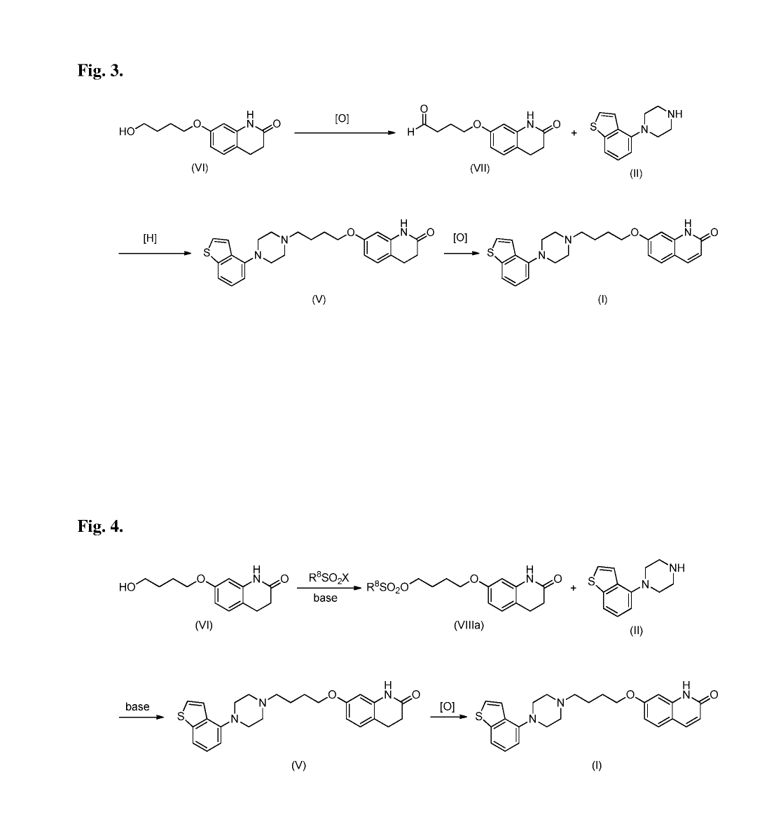

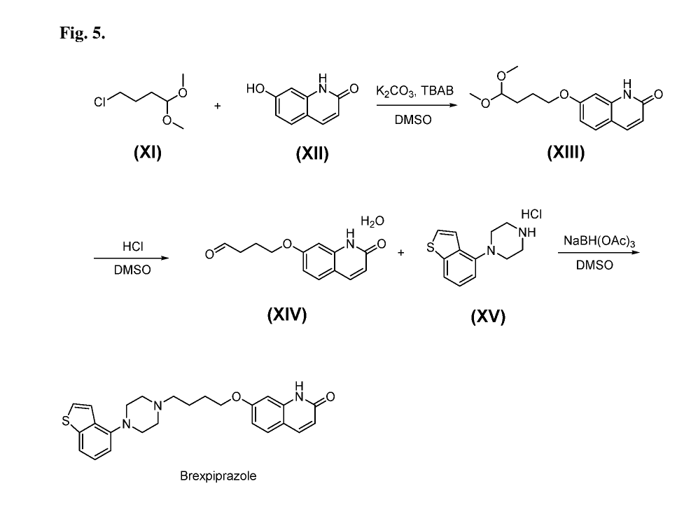

The present invention provides novel, efficient processes for preparing brexpiprazole from commercially available materials, as summarized in FIG. 3, FIG. 4, and FIG. 5. The present disclosure also provides a novel process for purifying brexpiprazole that provides brexpiprazole with superior purity and greatly improves the industrial applicability by reducing the time and difficulty of previously known purification schemes. In some embodiments, the process for preparing brexpiprazole may be coupled with the process for purification to produce a high yield and high purity brexpiprazole.

While a complete synthetic scheme is provided in the FIGS. 3, 4, and 5, one of skill in the art will appreciate that selected steps of the instant processes are novel and can be performed independent of the origin of the starting material or intermediates.

### II. Definitions

As used herein, the term “brexpiprazole” refers to 7-{4-[4-(1-benzothiophen-4-yl)piperazin-1-yl]butoxy}quinolin-2(1H)-one:

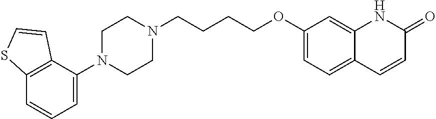 [SMILES: O=c1ccc2ccc(OCCCCN3CCN(c4cccc5sccc45)CC3)cc2[nH]1]

and salts thereof. Brexpiprazole is registered under CAS Registry No. 913611-97-9 and marketed under trade names including REXULTI. Brexpiprazole is described in U.S. Pat. Nos. 7,888,362; 8,349,840; and 8,618,109.

As used herein, the term “converting” refers to reacting a starting material with at least one reagent to form an intermediate species or a product. Converting includes reacting an intermediate with at least one reagent to form a further intermediate species or a product.

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents that can be produced in the reaction mixture.

As used herein, the term “oxidizing agent” refers to a reagent which can accept electrons from a substrate compound in an oxidation-reduction reaction. Electrons can be transferred from the substrate compound to the oxidizing agent in a process that includes addition of oxygen to the substrate compound or removal of hydrogen from the substrate compound. Examples of oxidizing agents include, but are not limited to, pyridinium chlorochromate, o-iodoxybenzoic acid, and 2,2,6,6-tetramethylpiperidine 1-oxyl.

As used herein, the term “nitroxyl-based reagent” refers to a substance having a nitroxide radical moiety (—N—O., wherein the dot represents an unpaired electron).

As used herein, the term “TEMPO” refers to 2,2,6,6-tetramethylpiperidine 1-oxyl.

As used herein, the term “reducing agent” refers to a reagent which can donate electrons to a substrate compound in an oxidation-reduction reaction. Electrons can be transferred from the reducing agent to the substrate compound in a process that includes addition of hydrogen to the substrate compound. Examples of reducing agents include, but are not limited to, sodium borohydride and sodium triacetoxyborohydride.

As used herein, the term “alkyl,” by itself or as part of another substituent, refers to a straight or branched chain hydrocarbon radical. Alkyl substituents, as well as other hydrocarbon substituents, may contain number designators indicating the number of carbon atoms in the substituent (i.e. C₁₋₈ means one to eight carbons), although such designators may be omitted. Unless otherwise specified, the alkyl groups of the present invention contain 1 to 12 carbon atoms. For example, an alkyl group can contain 1-2, 1-3, 1-4, 1-5, 1-6, 1-7, 1-8, 1-9, 1-10, 1-11, 1-12, 2-3, 2-4, 2-5, 2-6, 3-4, 3-5, 3-6, 4-5, 4-6 or 5-6 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

As used herein, the terms “halo” and “halogen,” by themselves or as part of another substituent, refer to a fluorine, chlorine, bromine, or iodine atom. Additionally, the term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

As used herein, the term “aryl,” by itself or as part of another substituent, refers to a polyunsaturated, typically aromatic, hydrocarbon group which can be a single ring or multiple rings (up to three rings) which are fused together or linked covalently. Examples of aryl groups include, but are not limited to, phenyl and naphthyl.

As used herein, the term “nitro,” by itself or as part of another substituent, refers to a moiety having the formula —NO₂.

As used herein, the term “base,” unless otherwise stated, refers to a Brønsted-Lowry base; that is, a substance capable of deprotonating a substrate compound when reacted with the substrate compound. Organic and inorganic bases can be used in the processes of the invention.

Examples of organic bases include, but are not limited to, Huenig's base (i.e., N,N-diisopropylethylamine), lutidines including 2,6-lutidine (i.e., 2,6-dimethylpyridine), triethylamine, and pyridine. Examples of inorganic bases include, but are not limited to, potassium carbonate and lithium hydroxide.

As used herein, the term “pharmaceutically acceptable excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the present invention include, but are not limited to, binders, fillers, disintegrants, lubricants, glidants, coatings, sweeteners, flavors and colors.

The term “phase transfer reagent” or “phase transfer catalyst” refers to a chemical compound which is soluble in both organic solvents and aqueous solvents. Without being bound to any particular theory, it is believe that phase transfer reagents facilitate the migration or transfer of a reactant from one phase to another phase. Phase transfer reagents can be useful in transferring reagents between phases such that all starting materials in a particular chemical conversion may contact one another. Phase transfer reagents can accelerate the rate of a reaction or can allow a reaction to proceed that would not otherwise occur. Typical phase-transfer catalysts include, but are not limited to, quarternary ammonium salts and phsophonium compounds. Once such non-limiting example of a phase transfer reagent is tetra-n-butylammonium bromide.

The term “chemically inert filtration bed” refers to a non-reactive chemical compound or composition that forms a selectively permeable layer which allows the chemically inert filtration bed to act as a filter. Substances in solution can pass through the selectively permeable layer of the chemically inert filtration bed, while solid components are retained or their movement is retarded when passing through the selectively permeable layer. In some instances a chemically inert filtration bed is more advantageous than typical filtration paper because it does not clog as easily during the filtration step. There are many known chemically inert filtration beds and their means of preparation are well established in the art. Non-limiting examples of chemically inert filtration beds include cellulose fiber, perlite, and celite, or a combination thereof.

### III. Embodiments of the Disclosure

A. Processes for the Preparation of Brexpiprazole

In one embodiment, provided herein is a process for preparing brexpiprazole, comprising:
(1) contacting a compound of Formula XIa

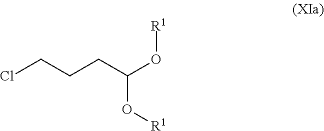

with a compound of Formula XII

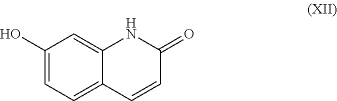 [SMILES: O=c1ccc2ccc(O)cc2[nH]1]

and a base in a first organic solvent to form a compound of Formula XIIIa;

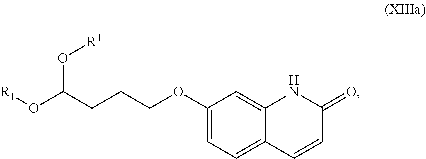

wherein each R¹ is independently C₁₋₈ alkyl, or optionally, the two R¹ moieties in combination with the oxygen atoms to which each are attached form a 5-8 membered heterocycloalkyl ring;
(2) contacting a compound of Formula XIIIa
   with an acid in a second organic solvent to form a compound of Formula XIV

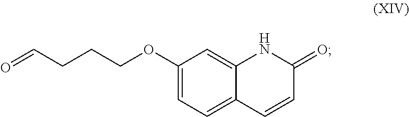 [SMILES: O=CCCCOc1ccc2ccc(=O)[nH]c2c1]

and
(3) contacting a compound of Formula XIV
   with a compound of Formula XV

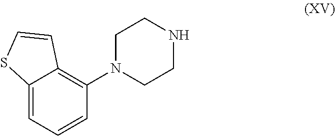 [SMILES: c1cc(N2CCNCC2)c2ccsc2c1]

or a salt thereof and a reducing agent in an organic solvent to form brexpiprazole.

As noted in the summary of the invention, a person of skill in the art will appreciate that selected steps in the process may be conducted independent of the origin of starting material or intermediates.

For example, in one embodiment, step (3) can be conducted independent of the process used to prepare the compound of Formula XIV. In this embodiment, brexpiprazole is prepared by
(3) contacting a compound of Formula XIV

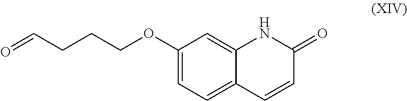 [SMILES: O=CCCCOc1ccc2ccc(=O)[nH]c2c1]

with a compound of Formula XV

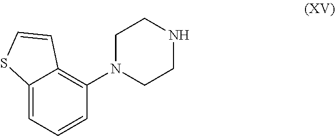 [SMILES: c1cc(N2CCNCC2)c2ccsc2c1]

or a salt thereof and a reducing agent in an organic solvent to form brexpiprazole.

The chemical conversion of step (3) includes a reductive amination where the compound of Formula XIV is reacted with the compound of Formula XV to form an intermediate of Formula XIVa

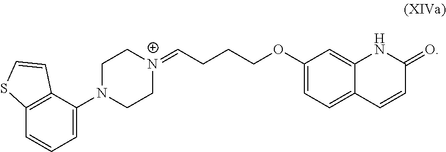 [SMILES: O=c1ccc2ccc(OCCCC=[N+]3CCN(c4cccc5sccc45)CC3)cc2[nH]1]

The intermediate is then reduced to provide the compound of Formula XIV. Accordingly, the compound of Formula XIV can be contacted with the compound of Formula XV in the presence of a reducing agent to form brexpiprazole. A variety of reducing agents can be used in the process. Examples of suitable reducing agents include, but are not limited to, sodium cyanoborohydride (NaCNBH₃); sodium borohydride (NaBH₄); sodium triacetoxyborohydride (NaBH(OAc)₃); 2-methylpyridine borane complex; decaborane (B₁₀H₁₄); and Et₃SiH; diethyl 1,4-dihydro-2,6-dimethyl-3,5-pyridinedicarboxylate (i.e., a Hantzsch ester). The reducing agents can be used with one or more additives including, but not limited to, acetic acid; trifluoroacetic acid; boric acid; InCl₃; thioruea; S-benzyl isothiouronium chloride; and palladium on carbon (Pd/C).

In some embodiments, step (3) further comprises
(3-i) contacting brexpiprazole, the organic solvent, and the reducing agent with an metal hydroxide to form solid brexpiprazole;
(3-ii) separating solid brexpiprazole to form isolated brexpiprazole.

The metal hydroxide in step (3-i) can be any suitable metal hydroxide which alters the pH sufficiently of the step (3) reaction to precipitate brexpiprazole. In some embodiments, the metal hydroxide is NaOH. In some embodiments a sufficient alteration in pH is a pH above 10.

The precipitation step described in step (3-i) can be aided by the alteration of temperature. In some embodiments, the solution of step (3-i) is heated. Heating can include temperatures from 40-80° C., 50-70° C., or 55-65° C. In some embodiments, the solution of step (3-i), after heating, is cooled to between 20-30° C. In some embodiments, water is added to the solution of step (3-i).

The compound of formula XV used in conversion (3) can be the free base of a salt thereof. For example, in some embodiments, the HCl salt of formula XV may be used

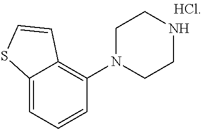 [SMILES: c1cc(N2CCNCC2)c2ccsc2c1]

It is contemplated that other salt forms may be used such as the HBr salt, the HI salt, the methanesulfonic acid (MsOH) salt, the p-Toluenesulfonic acid (TsOH) salt, acietic acid salt, or combinations thereof. Based on the description provided herein, a person of skill in the art could readily determine various appropriate salts.

The separation of solid brexpiprazole can be employed using a variety of techniques known in the art. In some embodiments, the separating step is a filtration step.

A number of organic solvents are suitable for the transformation described in step (3). In some embodiments, dimethyl sulfoxide (DMSO), dimethylacetamide (DMAc), dichloromethane, acetone, or a combination thereof are used. In some embodiments, the organic solvent is DMSO.

The process described in step (3) can produce brexpiprazole with high yield and purity. In some embodiments, the yield of step (3) is greater than 85%, 90%, 95%, or 97% (mol/mol) relative to Formula XIV. In some embodiments the purity of brexpiprazole produced is at least 80%, at least 85%, at least 90%, or at least 94%.

In a select group of embodiment, the process for preparing brexpiprazole utilizes the compound of Formula XIV, which can be prepared by
(2) contacting a compound of Formula XIIIa

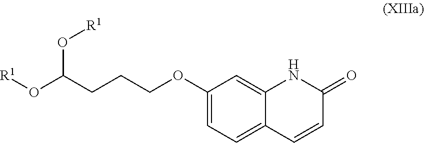

with an acid in a second organic solvent to form a compound of Formula XIV,
   wherein each R¹ is independently C₁₋₈ alkyl, or optionally, both R¹ moieties in combination with the oxygen atoms to which each are attached form a 5-8 membered heterocycloalkyl ring.

A number of acids are suitable for use in the conversion described in step (2). Some useful acids include HCl, HBr, HI, H₂SO₄, H₃PO₄, and acetic acid. In some embodiments, the acid is HCl.

The amount of acid necessary to convert the hemiacetal to an aldehyde will depend on the identity of the XIIIa compound, the identity of the acid, as well as the total time the reaction is allowed to incubate with the acid. In some embodiments, the final concentration of acid in the solution of step (2) is from 5-40% (v/v) or from 5-20% (v/v). In some embodiments, the final concentration of acid in the solution of step (2) is about 10% (v/v)

There are also a number of organic solvents that are useful in step (2). Some suitable solvents include dimethyl sulfoxide (DMSO), dimethylacetamide (DMAc), dichloromethane, and acetone. In some embodiments, the second organic solvent is DMSO.

In some embodiments, the compound of Formula XIIIa is the compound of Formula XIII

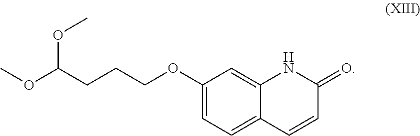 [SMILES: COC(CCCOc1ccc2ccc(=O)[nH]c2c1)OC]

In some embodiments, step (2) provides at least a 90 or 95% yield (mol/mol) with a purity of at least 90%, or at least 94%.

The reaction creating the compound of Formula XVI uses the compound of Formula XIIIa, which in some embodiments, can be prepared by
(1) contacting a compound of Formula XIa

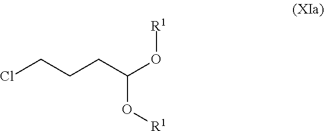

with a compound of Formula XII

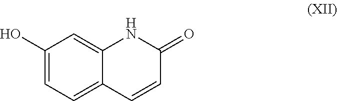 [SMILES: O=c1ccc2ccc(O)cc2[nH]1]

and a base in a first organic solvent to form a compound of Formula XIIIa

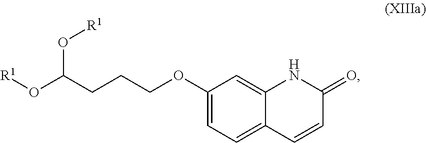

wherein R¹ is as defined previously.

The conversion in step (1) provides the coupling of compound XIa and XII. In this chemical coupling, a base is added—typically a base selected from Li₂CO₃, K₂CO₃, Cs₂CO₃, Na₂CO₃, NaHCO₃, and KHCO₃. In some embodiments the base is K₂CO₃ or Na₂CO₃.

A number of organic solvents are suitable for use in the conversion descried in step (1). Organic solvents useful in this conversion include, but are not limited to dimethyl sulfoxide (DMSO), dimethylacetamide (DMAc), dichloromethane, and acetone. In some embodiments, the first organic solvent is DMSO or DMAc.

In some embodiments, a phase transfer reagent is optionally included in the solution of step (1). The inclusion of the phase transfer reagent can increase the rate of the reaction, the yield, and/or the purity. In some embodiments, the phase transfer reagent is an ammonium salt, a phosphonium salt, or a combination thereof. In some embodiments the ammonium salt can be tetra-n-butylammonium bromide (TBAB), tetra-n-butylammonium iodide, tetra-n-butylammonium bisulfate, tetra-n-butylammonium cyanate, tetra-n-butylammonium methoxide, or tetra-n-butylammonium nitrate, or a combination thereof. In some embodiments, the phase transfer reagent is TBAB.

The conversion described in step (1) can be carried out at an elevated temperature. In some embodiments, the solution of step (1) is heated before the addition of the compound of Formula XII. The heating can include temperatures ranging from 50-90° C. or 60-70° C.

In some embodiments, EtOH is added to the heated reaction mixture. Useful forms of EtOH can include a 85-99.9%, 90-99.9%, or about 95% EtOH solution (v/v, with water).

In some embodiments, the mixture produced in step (1) is cooled to about 40° C. as EtOH and water is added. In some embodiments, after the addition of EtOH and water the reaction solution is further cooled to 0-10° C. In some embodiments, a precipitate is formed which can be isolated using filtration.

In some embodiments the compound of Formula XIa is represented by a compound of Formula XI

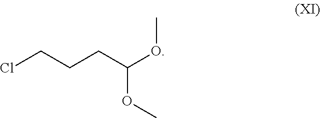

The conversion described in step (1) is advantageous because it is more selective that previously described methods. It provides a purity and yield that that is very desirable for synthetic purposes and very little starting material is lost. Additionally, by using a compound of Formula XII as a starting material, there is no necessary oxidation step involved DDQ. DDQ is an expensive reagent and its chemical by-product is difficult remove from the desired reaction products. Step (1) advantageously avoids these issues along with providing superior selectivity and activity.

In some embodiments, step (1) provides at least a 90% or at least a 94% yield (mol/mol) with a purity of at least 85 or 90%.

Further extraction, purification, and isolation steps can be performed after any of the described chemical conversions using any known means in the art including liquid-liquid extraction, column chromatography, crystallization, and filtration.

Processes for the preparation of brexpiprazole described herein may be coupled with particular steps in the process for the purification of brexpiprazole described in section B of the instant specification.

For example, in some embodiments, the preparation of brexpiprazole can include the preparation of a brexpiprazole acid salt comprising
(4) contacting brexpiprazole with a protic acid in an aqueous solution to form a brexpiprazole acid salt of Formula XVI

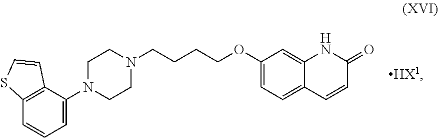

wherein X¹ is the anion of a protic acid.

Once the brexpiprazole acid salt is formed the process optionally includes
(5) contacting the brexpiprazole acid salt with activated carbon;
(6) filtering the brexpiprazole acid salt and the activated carbon through a chemically inert filtration bed to produce a purified brexpiprazole acid salt.

After forming the purified brexpiprazole acid salt, the process optionally includes
(7) contacting the purified brexpiprazole acid salt with a base in a second aqueous solution to form purified brexpiprazole.

A person of skill in the art will recognize that further purification of brexpiprazole embodiments described in section B may also be incorporated into the process for preparing brexpiprazole.

In another aspect, the invention provides a process for preparing brexpiprazole. The process includes converting a compound of Formula VI

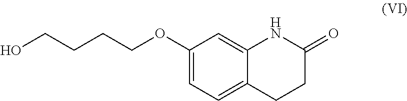 [SMILES: O=C1CCc2ccc(OCCCCO)cc2N1]

to brexpiprazole.

In some embodiments, converting the compound of Formula VI to brexpiprazole includes:
a) converting the compound of Formula VI to a compound of Formula VII

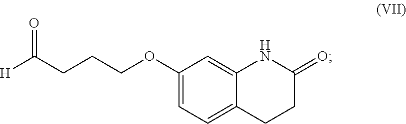 [SMILES: O=CCCCOc1ccc2c(c1)NC(=O)CC2]

b) contacting the compound of Formula VII with a compound of Formula II

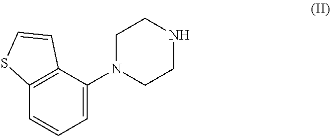 [SMILES: c1cc(N2CCNCC2)c2ccsc2c1]

to form a compound of Formula V
and

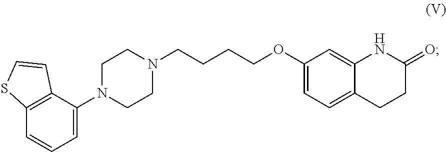 [SMILES: O=C1CCc2ccc(OCCCCN3CCN(c4cccc5sccc45)CC3)cc2N1]

c) converting the compound of Formula V to brexpiprazole.

In some embodiments, converting the compound of Formula VI to the compound of Formula VII includes contacting the compound of Formula VI with an oxidizing agent to form the compound of Formula VII.

Any oxidizing agent suitable for converting the compound of Formula VI to the compound of Formula VII can be used in the process of the invention. Examples of suitable oxidizing agents include, but are not limited to, chromium-based reagents (e.g., chromic acid; Jones reagent—chromium trioxide in aqueous sulfuric acid; Collins reagent—chromium trioxide pyridine complex; pyridinium dichromate; pyridinium chlorochromate and the like); dimethyl sulfoxide (DMSO)-based reagents (e.g., DMSO/oxalyl chloride; DMSO/diycyclohexyl-carbodiimide; DMSO/acetic anhydride; DMSO/phosphorous pentoxide; DMSO/trifluoroacetic anhydride; and the like); hypervalent iodine compounds (e.g., Dess-Martin periodinane; o-iodoxybenzoic acid; and the like); ruthenium-based reagents (e.g., ruthenium tetroxide; tetra-n-propylammonium perruthenate; and the like); and nitroxyl-based reagents (e.g., TEMPO-2,2,6,6-tetramethylpiperidine 1-oxyl—employed with sodium hypochlorite, bromine, or the like).

In some embodiments, the oxidizing agent is selected from a chromium-based reagent, a dimethyl sulfoxide-based reagent, a hypervalent iodine compound, a ruthenium-based reagent, and a nitroxyl-based reagent. In some embodiments, the oxidizing agent includes a nitroxyl-based reagent. Examples of nitroxyl-based reagents include, but are not limited to, TEMPO; NHAc-TEMPO; 4-C₁₋₆-alkyloxy-TEMPO; 4-hydroxy-TEMPO; diphenylnitroxyl; di-tert-butylnitroxyl; 9-azabicyclo[3.3.1]nonane N-oxyl (ABNO); and 2-azaadamantane N-oxyl (AZADO). In some embodiments, the oxidizing agent includes TEMPO.

The nitroxyl-base reagent can be used in conjunction with oxidants including, but not limited to, sodium hypochlorite, bromine, potassium bromide, sodium bromide, and iodobenzene I,I-diacetate. Oxidation reactions can be conducted with the nitroxyl-based reagent using molecular oxygen as the terminal oxidant; such reactions can be conducted in the presence of additional additives including, but not limited to, sodium nitrite, sodium nitrate, nitric acid, and hydrochloric acid. Oxidation reactions conducted with the nitroxyl-based reagent and molecular oxygen can be catalyzed using metals salts or complexes, such as copper salts and copper complexes. Examples of metal salts suitable for use with the nitroxyl-based reagents include, but are not limited to, Cu(OTf)(MeCN)₄; CuBr; CuI; Cu(OTf)₂; Cu(TFA)₂; Pd(OAc)₂; and Fe(NO₃)₃. Oxidation reactions conducted with nitroxyl-based reagents and metal salts can further include one or more additional components including, but not limited to, 2,2′-bipyridine; 4,4′-di-tert-butyl bipyridine; 2,9-dimethyl-1,10-phenanthroline; 2,2′-ethylenebis(nitrilomethylidene)-diphenol; potassium carbonate; sodium hydroxide; sodium chloride; N-methylimidazole; and 1,4-diazabicyclo[2.2.2]octane.

In some embodiments, the process for preparing brexpiprazole includes contacting the compound of Formula VI with an oxidizing agent includes TEMPO, sodium hypochlorite, and sodium bicarbonate, to form the compound of Formula VII.

Any solvent suitable for forming the compound of Formula VII can be used for the oxidation step in the process of the invention. Examples of suitable solvents include, but are not limited to, water, methanol, ethanol, isopropyl alcohol, isobutyl alcohol, 1,4-dioxane, dichloromethane, toluene, tetrahydrofuran (THF), 2-methyl-tetrahydrofuran (2-Me-THF), diglyme, acetonitrile, N-methylpyrrolidone (NMP), N,N-dimethylformamide (DMF), N,N-dimethylacetamide (DMAC), ethylene glycol and combinations thereof. The oxidation step can be conducted in a biphasic mixture containing an organic phase (e.g., toluene) and an aqueous phase (e.g., water or aqueous sodium bicarbonate). The compound of Formula VI and other reagents used in the oxidation step can be dissolved in (or otherwise combined with) the solvent at any suitable concentration (e.g., around 100 μM, or around 1 mM, or around 10 mM, or around 100 mM). The oxidation step can be conducted at any temperature suitable for forming the compound of Formula VII. In general, the oxidation step is conducted at a temperature ranging from about −78° C. to about 60° C. (e.g., about 0° C., or about 4° C., or about 25° C., or about 40° C.). The oxidation step can be conducted for any amount of time necessary to form the compound of Formula VII. The oxidation step can be conducted, for example, over a period of time ranging from a few minutes to several hours. One of skill in the art will appreciate that the length of time will depend on factors including the solvent and temperature used for the oxidation step, as well as the particular oxidizing agent.

As described above, certain embodiments of the invention provide a process for preparing brexpiprazole that includes contacting a compound of Formula VII

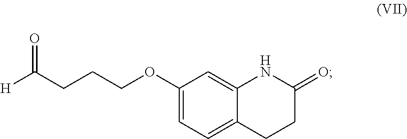 [SMILES: O=CCCCOc1ccc2c(c1)NC(=O)CC2]

with a compound of Formula II

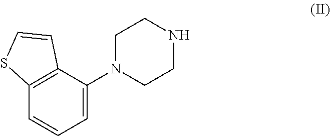 [SMILES: c1cc(N2CCNCC2)c2ccsc2c1]

to form a compound of Formula V

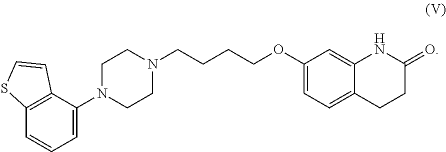 [SMILES: O=C1CCc2ccc(OCCCCN3CCN(c4cccc5sccc45)CC3)cc2N1]

In this reductive amination step, the compound of Formula VII is reacted with the compound of Formula II to form an intermediate of Formula VIIa

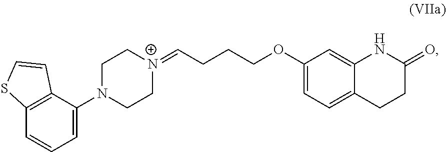 [SMILES: O=C1CCc2ccc(OCCCC=[N+]3CCN(c4cccc5sccc45)CC3)cc2N1]

and the intermediate is reduced to provide the compound of Formula V. Accordingly, the compound of Formula VII can be contacted with the compound of Formula II in the presence of a reducing agent, so as to form the compound of Formula V. Any reducing agent suitable for forming the compound of Formula V can be used in the process of the invention. Examples of suitable reducing agents include, but are not limited to, sodium cyanoborohydride (NaCNBH₃); sodium borohydride (NaBH₄); sodium triacetoxyborohydride (NaBH(OAc)₃); 2-methylpyridine borane complex; decaborane (B₁₀H₁₄); and Et₃SiH; diethyl 1,4-dihydro-2,6-dimethyl-3,5-pyridinedicarboxylate (i.e., a Hantzsch ester). The reducing agents can be used with one or more additives including, but not limited to, acetic acid; trifluoroacetic acid; boric acid; InCl₃; thioruea; S-benzyl isothiouronium chloride; and palladium on carbon (Pd/C).

In some embodiments, the reducing agent is selected from sodium cyanoborohydride, sodium borohydride, sodium triacetoxyborohydride, and 2-methylpyridine borane complex. In some embodiments, the reducing agent is sodium triacetoxyborohydride (i.e., NaBH(OAc)₃). In some embodiments, the compound of Formula VII is contacted with the compound of Formula II in the presence of sodium triacetoxyborohydride and acetic acid.

Any solvent suitable for forming the compound of Formula V can be used for the reductive amination step in the process of the invention. Examples of suitable solvents include, but are not limited to, water, methanol, ethanol, isopropyl alcohol, isobutyl alcohol, 1,4-dioxane, dichloromethane, toluene, tetrahydrofuran (THF), 2-methyl-tetrahydrofuran (2-Me-THF), diglyme, acetonitrile, N-methylpyrrolidone (NMP), N,N-dimethylformamide (DMF), N,N-dimethylacetamide (DMAC), ethylene glycol, acetic acid, trifluoroacetic acid, and combinations thereof. In some embodiments, the reductive amination step is conducted in mixture containing methanol and acetic acid. The compounds of Formula VII and Formula II and other reagents used in the reductive amination step can be dissolved in (or otherwise combined with) the solvent at any suitable concentration (e.g., around 100 μM, or around 1 mM, or around 10 mM, or around 100 mM). The reductive amination step can be conducted at any temperature suitable for forming the compound of Formula V. In general, the reductive amination step is conducted at a temperature ranging from about −78° C. to about 60° C. (e.g., about 0° C., or about 4° C., or about 25° C., or about 40° C.). The reductive amination step can be conducted for any amount of time necessary to form the compound of Formula V. The reductive amination step can be conducted, for example, over a period of time ranging from a few minutes to several hours. One of skill in the art will appreciate that the length of time will depend on factors including the solvent and temperature used for the reductive amination step, as well as the particular reducing agent.

In some embodiments, converting the compound of Formula VI to brexpiprazole includes:
i) converting the compound of Formula VI to a compound of Formula VIII

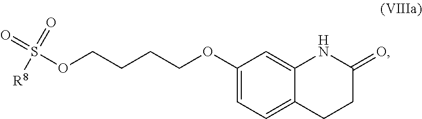

wherein R⁸ is selected from C₁₋₆ alkyl, C₁₋₆ haloalkyl, and C₆₋₁₀ aryl, wherein C₆₋₁₀ aryl is optionally substituted with one or more substituents independently selected from halo and nitro;
ii) contacting the compound of Formula VIII with a compound of Formula II

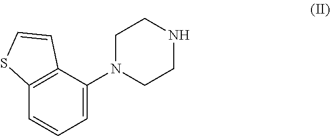 [SMILES: c1cc(N2CCNCC2)c2ccsc2c1]

to form a compound of Formula V
and

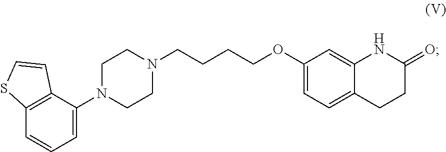 [SMILES: O=C1CCc2ccc(OCCCCN3CCN(c4cccc5sccc45)CC3)cc2N1]

iii) converting compound (V) to brexpiprazole.

In some embodiments, converting the compound of Formula VI to the compound of Formula VIIIa includes esterifying the compound of Formula VI with a compound of Formula VIIIb

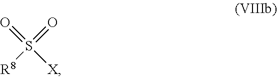

wherein R⁸ is selected from C₁₋₆ alkyl, C₁₋₆ haloalkyl, and C₆₋₁₀ aryl, wherein C₆₋₁₀ aryl is optionally substituted with one or more substituents independently selected from halo and nitro; and X is selected from —OH, halo, and —O(SO₂)R⁸;
under conditions to sufficient to form the compound of Formula VIIIa.

In some embodiments, R⁸ is selected from methyl, trifluoromethyl, 4-methylphenyl, 4-bromophenyl, and 4-nitrophenyl. In some embodiments, R⁸ is methyl.

In some embodiments, X is halo. In some embodiments, X is chloro.

In some embodiments, R⁸ is selected from methyl, trifluoromethyl, 4-methylphenyl, 4-bromophenyl, and 4-nitrophenyl; and X is halo. In some embodiments, R⁸ is selected from methyl, trifluoromethyl, 4-methylphenyl, 4-bromophenyl, and 4-nitrophenyl; and X is chloro. In some embodiments, R⁸ is methyl and X is halo. In some embodiments, R⁸ is methyl and X is chloro.

In some embodiments, the compound of Formula VI is contacted with a compound of Formula VIIIb in the presence of a base. Any base suitable for converting the compound of Formula VI to the compound VIIIa can be used in this step of the process. Examples of suitable bases include, but are not limited to, Huenig's base (i.e., N,N-diisopropylethylamine), lutidines including 2,6-lutidine (i.e., 2,6-dimethylpyridine), triethylamine, tributylamine, pyridine, 2,6-di-tert-butylpyridine, 1,8-diazabicycloundec-7-ene (DBU), 1,5,7-triazabicyclo(4.4.0)dec-5-ene (TBD), 7-methyl-1,5,7-triazabicyclo(4.4.0)dec-5-ene (MTBD), 1,5-diazabicyclo[4.3.0]non-5-ene (DBN), 1,1,3,3-tetramethylguanidine (TMG), 2,2,6,6-tetramethylpiperidine (TMP), pempidine (PMP), 1,4-diazabicyclo[2.2.2]octane (TED), quinuclidine, and the collidines.

In some embodiments, the process of the invention includes contacting the compound of Formula VI with a compound of Formula VIIIb in the presence of a base to from the compound of Formula VIIIa, wherein the base is selected from N,N-diisopropylethylamine; 2,6-lutidine; triethylamine; tributylamine; pyridine; 2,6-di-tert-butylpyridine; 1,8-diazabicycloundec-7-ene; 1,5,7-triazabicyclo(4.4.0)dec-5-ene; 7-methyl-1,5,7-triazabicyclo(4.4.0)dec-5-ene; 1,5-diazabicyclo[4.3.0]non-5-ene; 1,1,3,3-tetramethylguanidine; 2,2,6,6-tetramethylpiperidine; pempidine; 1,4-diazabicyclo[2.2.2]octane; and quinuclidine. In some embodiments, the base is triethylamine.

In some embodiments, the compound of Formula VIIIa is a compound of Formula VIII:

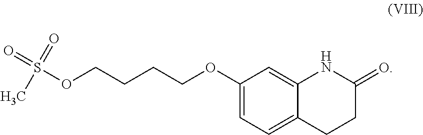 [SMILES: CS(=O)(=O)OCCCCOc1ccc2c(c1)NC(=O)CC2]

In some such embodiments, the process of includes contacting the compound of Formula VI with the compound of Formula VIIIb, wherein R⁸ is methyl and X is chloro, in the presence of triethylamine to form the compound of Formula VIII.

Any solvent suitable for forming the compound of Formula VIIIa can be used for the esterification step in the process of the invention. Examples of suitable solvents include, but are not limited to, water, methanol, ethanol, isopropyl alcohol, isobutyl alcohol, 1,4-dioxane, dichloromethane, toluene, tetrahydrofuran (THF), 2-methyl-tetrahydrofuran (2-Me-THF), diglyme, acetonitrile, N-methylpyrrolidone (NMP), N,N-dimethylformamide (DMF), N,N-dimethylacetamide (DMAC), ethylene glycol, and combinations thereof. In some embodiments, the esterification step is conducted using dichloromethane as the solvent. The compounds of Formula VI and Formula VIIIb and other reagents used in the esterification step can be dissolved in (or otherwise combined with) the solvent at any suitable concentration (e.g., around 100 μM, or around 1 mM, or around 10 mM, or around 100 mM). The esterification step can be conducted at any temperature suitable for forming the compound of Formula VIIIa. In general, the esterification step is conducted at a temperature ranging from about −78° C. to about 60° C. (e.g., about 0° C., or about 4° C., or about 25° C., or about 40° C.). The esterification step can be conducted for any amount of time necessary to form the compound of Formula VIIIa. The esterification step can be conducted, for example, over a period of time ranging from a few minutes to several hours. One of skill in the art will appreciate that the length of time will depend on factors including the solvent and temperature used for the esterification step, as well as the identity of the R⁸ group and the X group in the compound of Formula VIIIb.

As described above, certain embodiments of the invention provide a process for preparing brexpiprazole that includes contacting a compound of Formula VIIIa

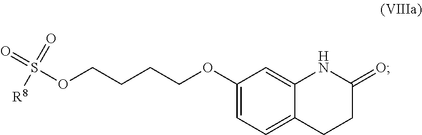

with a compound of Formula II

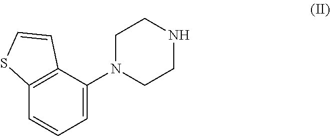 [SMILES: c1cc(N2CCNCC2)c2ccsc2c1]

to form a compound of Formula V

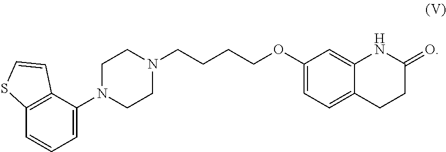 [SMILES: O=C1CCc2ccc(OCCCCN3CCN(c4cccc5sccc45)CC3)cc2N1]

Typically, the compound of Formula VIIIa is contacted with the compound of Formula II in the presence of a base. Any base suitable for forming the compound of Formula V can be used in the reaction. Examples of suitable bases include, but are not limited to, sodium bicarbonate, sodium carbonate, potassium carbonate, lithium hydroxide, potassium hydroxide, cesium hydroxide, and cesium fluoride. In some embodiments, the base is potassium carbonate.

Any solvent suitable for forming the compound of Formula V can be used for the alkylation step in the process of the invention. Examples of suitable solvents include, but are not limited to, water, methanol, ethanol, isopropyl alcohol, isobutyl alcohol, 1,4-dioxane, dichloromethane, toluene, tetrahydrofuran (THF), 2-methyl-tetrahydrofuran (2-Me-THF), diglyme, acetonitrile, N-methylpyrrolidone (NMP), N,N-dimethylformamide (DMF), N,N-dimethylacetamide (DMAC), ethylene glycol, and combinations thereof. In some embodiments, the alkylation step is conducted using acetonitrile as the solvent. The compounds of Formula VIIIa and Formula II and other reagents used in the alkylation step can be dissolved in (or otherwise combined with) the solvent at any suitable concentration (e.g., around 100 μM, or around 1 mM, or around 10 mM, or around 100 mM). The alkylation step can be conducted at any temperature suitable for forming the compound of Formula V. In general, the alkylation step is conducted at a temperature ranging from about −78° C. to about 60° C. (e.g., about 0° C., or about 4° C., or about 25° C., or about 40° C.). The alkylation step can be conducted for any amount of time necessary to form the compound of Formula V. The alkylation step can be conducted, for example, over a period of time ranging from a few minutes to several hours. One of skill in the art will appreciate that the length of time will depend on factors including the solvent and temperature used for the alkylation step, as well as the identity of the R⁸ group in the compound of Formula VIIIa.

In some embodiments, converting the compound of Formula V to brexpiprazole includes contacting the compound of Formula V with an oxidizing agent to form brexpiprazole.

In some embodiments, the oxidizing agent is selected from 1,4-benzoquinone; 2,3-dichloro-5,6-dicyanobenzoquinone (DDQ); and 3,3′,5,5′-tetra-tert-butyldiphenoquinone (DPQ). In some embodiments, the oxidizing agent is DDQ.

Any solvent suitable for forming brexpiprazole (I) can be used for the dehydrogenation step in the process of the invention. Examples of suitable solvents include, but are not limited to, water, methanol, ethanol, isopropyl alcohol, isobutyl alcohol, 1,4-dioxane, dichloromethane, toluene, tetrahydrofuran (THF), 2-methyl-tetrahydrofuran (2-Me-THF), diglyme, acetonitrile, N-methylpyrrolidone (NMP), N,N-dimethylformamide (DMF), N,N-dimethylacetamide (DMAC), ethylene glycol, and combinations thereof. In some embodiments, the dehydrogenation step is conducted using THF as the solvent. The compound of Formula V and other reagents used in the dehydrogenation step can be dissolved in (or otherwise combined with) the solvent at any suitable concentration (e.g., around 100 μM, or around 1 mM, or around 10 mM, or around 100 mM). The dehydrogenation step can be conducted at any temperature suitable for forming the brexpiprazole. In general, the dehydrogenation step is conducted at a temperature ranging from about −78° C. to about 60° C. (e.g., about 0° C., or about 4° C., or about 25° C., or about 40° C.). The dehydrogenation step can be conducted for any amount of time necessary to form brexpiprazole. The dehydrogenation step can be conducted, for example, over a period of time ranging from a few minutes to several hours. One of skill in the art will appreciate that the length of time will depend on factors including the solvent and temperature used for the dehydrogenation step, as well as the particular oxidizing agent.

In another aspect, the invention provides brexpiprazole prepared according to the process as described above. In a related aspect, the invention provides a pharmaceutical composition containing brexpiprazole prepared according to the process as described above and a pharmaceutically acceptable excipient.

B. Processes for the Purification of Brexpiprazole

In one embodiment, provided herein is a process for purifying brexpiprazole, comprising:
(a) contacting brexpiprazole with a protic acid in a first aqueous solution to form a brexpiprazole acid salt of Formula XVI

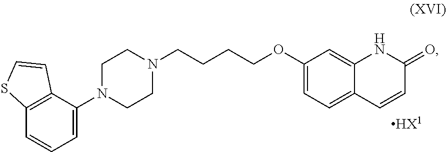

wherein X¹ is the anion of a protic acid;
(b) contacting the brexpiprazole acid salt with activated carbon;
(c) filtering the brexpiprazole acid salt and the activated carbon through a chemically inert filtration bed to produce a purified brexpiprazole acid salt;
(d) contacting the purified brexpiprazole acid salt with a base in a second aqueous solution to form purified brexpiprazole.

A variety of protic acids are suitable in the formation of Formula XVI shown in step (a). Exemplary acids include, but are not limited to HCl, HBr, HI, H₂SO₄, H₃PO₄, acetic acid, HNO₃, H₂SO₃, para-toluenesulfonic acid (pTsOH, or TsOH), and methansulfonic acid. In some embodiments, the protic acid is HCl.

The anion of the protic acid (X¹) of Formula XVI will vary depending on the protic acid used. As such, suitable anions include chloride, bromide, iodide, sulfonate, tosylate, mesylate, nitrate and acetate, or combinations thereof. In some embodiments, the anion of the protic acid (X¹) of Formula XVI is chloride.

The amount of protic acid added will depend on the protic acid used. In some embodiments, the amount of protic acid added is an amount sufficient to adjust the pH of the aqueous solution to less than 3.

In some embodiments, the first aqueous solution of step (a) further comprises isopropyl alcohol. Other lower alkyl alcohols are also suitable for use in the first aqueous solution.

In some embodiments, step (a) further comprises
(a-iii) separating the brexpiprazole acid salt to form an isolated brexpiprazole acid salt,
   wherein step (a-iii) is performed after contacting brexpiprazole with the protic acid in the aqueous solution.

The separation can include any known means in the art to isolate a solid from the remaining solution including, but not limited to, centrifugation and filtration. In some embodiments, the separation step is a filtering step.

The formation of the brexpiprazole acid salt may be aided by varying the temperature after contacting brexpiprazole with the protic acid. Therefore, in some embodiments step (a) further comprises
(a-i) heating the aqueous solution to about 50-70° C.;
(a-ii) cooling the aqueous solution to about 10-40° C.,
   wherein step (a-i)-(a-ii) are performed before said separating step (a-iii).

In some embodiments, the heating and cooling steps of (a-i) and (a-ii) are conducted at temperatures of from 55-65° C. and 20-30° C., respectively.

Contacting the brexpiprazole acid salt with activated carbon will aide in the removal of impurities present.

The chemically inert filtration bed used in step (c) is useful in removing the activated carbon from the suspension. There are many known chemically inert filtration beds that are useful in the present process. In some embodiments, the chemically inert filtration bed is cellulose fiber, perlite, or celite. In some embodiments, the chemically inert filtration bed is celite.

The conversion of the purified brexpiprazole acid salt to brexpiprazole can be achieved by admixing the purified brexpiprazole acid salt with a base in an aqueous solution. Accordingly, step (d) comprises
(d) contacting the purified brexpiprazole acid salt with a base in a second aqueous solution to form purified brexpiprazole.

A variety of different bases are useful in step (d). Typical suitable bases include metal hydroxides. In some embodiments the base is NaOH.

In some embodiments, the second aqueous solution of step (d) further comprises isopropyl alcohol. Other lower alkyl alcohols are also suitable for use in the second aqueous solution.

In some embodiments, step (d) further comprises
(d-iii) isolating purified brexpiprazole from the second aqueous solution,
   wherein step (d-iii) is performed after contacting the brexpiprazole acid salt with the second base in the second aqueous solution.

The isolating can include any known means in the art to separate a solid from the remaining solution including, but not limited to, centrifugation and filtration. In some embodiments, the isolating is carried out by filtering the second aqueous solution after addition of the second base.

The formation of purified brexpiprazole may be aided by varying the temperature after contacting purified brexpiprazole acid salt with the base. Therefore, in some embodiments step (d) further comprises
(d-i) heating the second aqueous solution to about 60-90° C.;
(d-ii) cooling the second aqueous solution to about 10-40° C.,
   wherein steps (d-i)-(d-ii) are performed before the isolating step (d-iii).

In some embodiments, the heating and cooling steps of (d-i) and (d-ii) are conducted at temperatures of from 55-65° C. and 20-30° C., respectively.

In some embodiments, step (a) provides at least an 85 or 90% yield (mol/mol) with a purity of at least 90%, or at least 95%.

In some embodiments, steps (b) and (c) provide at least an 85% or 90% yield (mol/mol) with a purity of at least 95%, or at least 97%.

In some embodiments step (c) proves at least a 90 or 95% yield (mol/mol) with a purity of at least 97 or 99%.

Compared to prior disclosed processes of making brexpiprazole, which used column chromatography, the instantly described purification process provides surprisingly pure brexpiprazole. In some embodiments the purity of the processes described herein provide at least 97%, 98%, 99%, or 99.5% pure brexpiprazole.

### IV. Examples

The following examples are provided to further illustrate, but not to limit this invention.

### Example 1: Preparation of Compound of Formula (XIII)

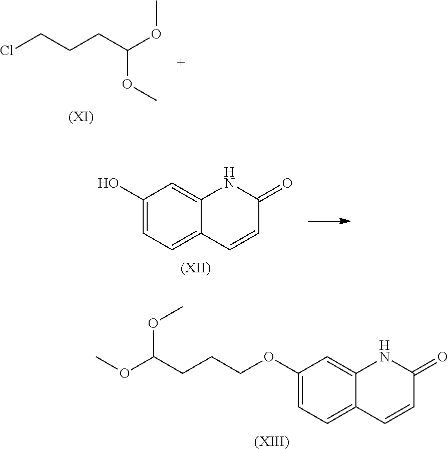

Compound of Formula (XI) (35 g), K₂CO₃ (30 g), TBAB (35 g), DMSO (245 mL), and Compound of Formula (XII) (39.8 g) were sequentially added to a suitable flask at 20-40° C. The mixture was heated to 60-70° C. and stirred for not longer than 20 hr. After the reaction was complete, H₂O was slowly added and the resulting solution was stirred for 1 hr. After an hour, additional H₂O was added. The mixture was then cooled to 0-10° C. and stirred for another 2 hr. The mixture was filtered and dried. Dry Compound of Formula (XIII) (56.5 g) was isolated in 93.8% yield with 83.26% purity.

### Example 2: Preparation of Compound of Formula (XIV)

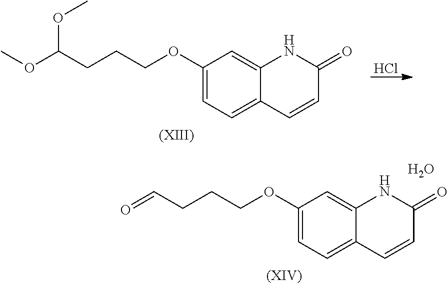

Compound of Formula (XIII) (43 g) and DMSO (258 mL) were added to a 1 L 4-necked round bottom flask equipped with a mechanical stirrer and a thermometer at 20-40° C. A solution of HCl (283 g) was added into the mixture at 40° C. and the mixture was stirred at 20-40° C. for 1 hr. After the reaction was completed, H₂O was added at 20-40° C. followed by cooling to 0-10° C. and the mixture was then stirred for 1 hr. The mixture was filtered. Dry compound of Formula (XIV) cake (37.6 g) was obtained in 97.4% yield with 95.45% purity as a pale-yellow solid.

### Example 3: Preparation of Brexpiprazole

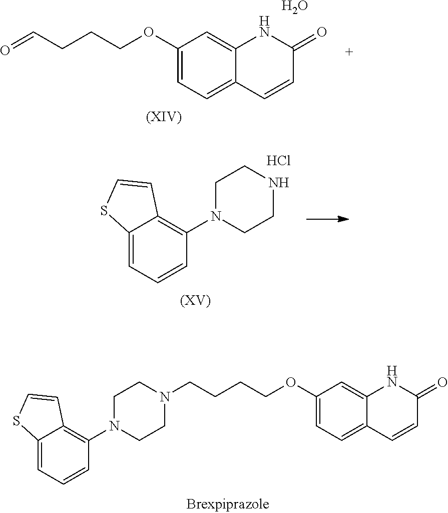

Compound of Formula (XIV) (34 g), compound of Formula (XV) (35.6 g) and DMSO (238 mL) were added to a 1 L 4-necked round bottom flask equipped with a mechanical stirrer and a thermometer at 20-40° C. NaBH(OAc)₃ (29.61 g) was added into the mixture. The mixture was then stirred for 1 hr. After the reaction was completed, NaOH (98.62 g) was added to adjust pH>10 at 20-40° C. The mixture was heated to 55-65° C., and then water was added. The mixture was cooled to 20-30° C. and stirred at that temperature for 2 hr. The mixture was filtered and dried. Dry Brexpiprazole (56.47 g) was obtained in 95.5% yield with 94.89% purity.

### Example 4: Preparation of HCl Salt of Brexpiprazole

Brexpiprazole (72 g), IPA (720 mL) and H₂O (360 mL) were added to a 2 L 4-necked round bottom flask equipped with a mechanical stirrer and a thermometer at 20-40° C. A solution of HCl (61.75 g) was added into the mixture at no more than 40° C. to adjust pH to <3, and the mixture was heated to 55-65° C. H₂O (360 mL) was added into the mixture at 55-65° C. and stirred for 1 hr. The mixture was cooled to 20-30° C. and stirred for 2 hr. The mixture was filtered and dried. HCl salt of Brexpiprazole (75.2 g) was isolated in 91.1% yield with 96.8% purity as a light yellow to off-white solid.

### Example 5: Purification of HCl Salt of Brexpiprazole

HCl salt of Brexpiprazole (74 g), EtOH (740 mL) and H₂O (592 mL) were added to a 2 L 4-necked round bottom flask equipped with a mechanical stirrer and a thermometer at 20-40° C. The mixture was heated to 70-80° C. and activated Carbon was added into the mixture. The mixture was heated to 75-85° C. and stirred at the temperature for 1 hr. The mixture was filtered at 75-85° C. with a celite bed and washed with hot EtOH/H₂O (148 mL, 2 vol). The mixture was heated to 75-85° C. for dissolution then cooled to 55-65° C. After being stirred for 1 hr, the mixture was further cooled to 20-30° C. and stirred for another 1 hr. The mixture was filtered and dried. Purified HCl salt of Brexpiprazole (67.85 g) was isolated in 91.7% yield with 99.0% purity as a light yellow to off-white solid.

### Example 6: Preparation of Purified Brexpiprazole

Purified HCl salt of Brexpiprazole (20 g), EtOH (200 mL), and water (160 mL) were added to a 2 L 4-necked round bottom flask equipped with a mechanical stirrer and a thermometer at 20-40° C. The mixture was heated to 70-80° C. for dissolution and NaOH (6.81 g) was added at about 70° C. into the mixture and stirred for 1 hr. The mixture was cooled to 20-30° C. and stirred for another 1 hr. The mixture was filtered and dried. Purified Brexpiprazole (17.78 g) was isolated in 96.1% yield with 99.71% purity as a white to off-white solid.

### Example 7: Preparation of brexpiprazole via reductive amination of 4-((2-oxo-1,2,3,4-tetrahydroquinolin-7-yl)oxy)butanal

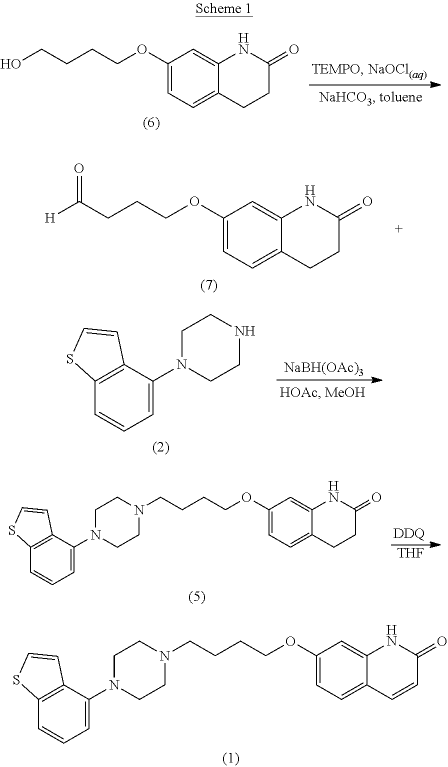

Brexpiprazole is prepared according to Scheme 1. 7-(4-hydroxybutoxy)-3,4-dihydroquinolin-2(1H)-one (6) is converted to 4-((2-oxo-1,2,3,4-tetrahydroquinolin-7-yl)oxy)butanal (7) using TEMPO and sodium hypochlorite in a biphasic mixture of toluene and aqueous sodium bicarbonate. 4-((2-Oxo-1,2,3,4-tetrahydroquinolin-7-yl)oxy)butanal (7) is reacted with 1-(benzo[b]thiophen-4-yl)piperazine (2) in a methanolic solution containing sodium triacetoxyborohydride and acetic acid to provide 7-(4-(4-(benzo[b]thiophen-4-yl)piperazin-1-yl)butoxy)-3,4-dihydroquinolin-2(1H)-one (5). 7-(4-(4-(Benzo[b]thiophen-4-yl)piperazin-1-yl)butoxy)-3,4-dihydroquinolin-2(1H)-one (5) is dissolved in THF and dehydrogenated with DDQ to obtain brexpiprazole (1) in good yield.

### Example 8: Preparation of brexpiprazole via substitution of 4-((2-oxo-1,2,3,4-tetrahydroquinolin-7-yl)oxy)butyl methanesulfonate

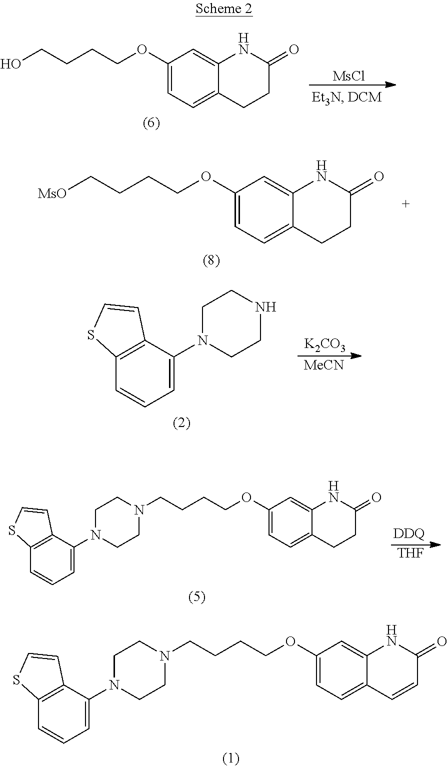

Brexpiprazole is prepared according to Scheme 2. 7-(4-hydroxybutoxy)-3,4-dihydroquinolin-2(1H)-one (6) is reacted with methanesulfonyl chloride in a solution of dichloromethane containing triethylamine to afford 4-((2-oxo-1,2,3,4-tetrahydroquinolin-7-yl)oxy)butyl methanesulfonate (8). 4-((2-Oxo-1,2,3,4-tetrahydroquinolin-7-yl)oxy)butyl methanesulfonate (8) is substituted with 1-(benzo[b]thiophen-4-yl)piperazine (2) in acetonitrile containing potassium carbonate to provide 7-(4-(4-(benzo[b]thiophen-4-yl)piperazin-1-yl)butoxy)-3,4-dihydroquinolin-2(1H)-one (5). 7-(4-(4-(Benzo[b]thiophen-4-yl)piperazin-1-yl)butoxy)-3,4-dihydroquinolin-2(1H)-one (5) is dissolved in THF and dehydrogenated with DDQ to obtain brexpiprazole (1) in good yield.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A process for preparing brexpiprazole 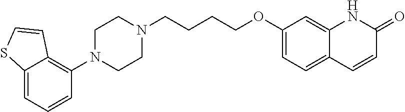 [SMILES: O=c1ccc2ccc(OCCCCN3CCN(c4cccc5sccc45)CC3)cc2[nH]1]
comprising
(3) contacting a compound of Formula XIV 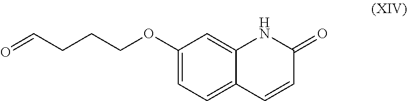 [SMILES: O=CCCCOc1ccc2ccc(=O)[nH]c2c1]
with a compound of Formula XV 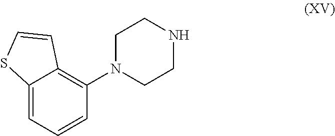 [SMILES: c1cc(N2CCNCC2)c2ccsc2c1]
or a salt thereof and a reducing agent in an organic solvent to form brexpiprazole.

2. The process of claim 1, wherein the compound of formula XV is the HCl salt

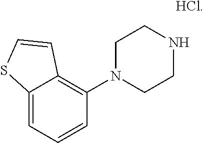

3. The process of claim 1, further comprising
(3-i) contacting brexpiprazole, the organic solvent, and the reducing agent with an metal hydroxide to form solid brexpiprazole;
(3-ii) separating solid brexpiprazole to form isolated brexpiprazole.

4. The process of claim 3, wherein the metal hydroxide is NaOH.

5. The process of claim 1, wherein the reducing agent is selected from the group consisting of sodium cyanoborohydride, sodium borohydride, sodium triacetoxy-borohydride, and 2-methylpyridine borane complex.

6. The process of claim 5, wherein the reducing agent is sodium triacetoxy-borohydride.

7. The process of claim 1, wherein the organic solvent is selected from the group consisting of dimethyl sulfoxide (DMSO), dimethylacetate (DMAc), dichloromethane, and acetone.

8. The process of claim 7, wherein the organic solvent is DMSO.

9. The process of claim 1, wherein the compound of Formula XIV is prepared by
(2) contacting a compound of Formula XIIIa 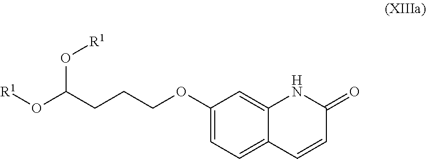
with an acid in a second organic solvent to form a compound of Formula XIV,
wherein each R¹ is independently C₁₋₁₈ alkyl, or both R¹ moieties in combination with the oxygen group to which they are attached may form a 5-8 membered heterocycloalkyl ring.

10. The process of claim 9, wherein the acid is selected from the group consisting of HCl, HBr, HI, H₂SO₄, H₃PO₄, and acetic acid.

11. The process of claim 10, wherein the acid is HCl.

12. The process of claim 11, wherein the final concentration of acid in step (2) is from 5-40% (v/v).

13. The process of claim 12, wherein the final concentration of acid in step (2) is from 5-20% (v/v).

14. The process of claim 13, wherein the final concentration of acid in step (2) is about 10% (v/v).

15. The process of claim 9, wherein the second organic solvent is selected from the group consisting of dimethyl sulfoxide (DMSO), dimethylacetate (DMAc), dichloromethane, and acetone.

16. The process of claim 15, wherein the second organic solvent is DMSO.

17. The process of claim 9, wherein the compound of Formula XIIIa is the compound of Formula XIII

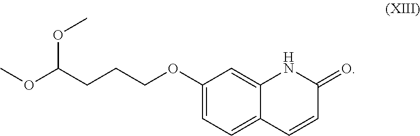 [SMILES: COC(CCCOc1ccc2ccc(=O)[nH]c2c1)OC]

18. The process of claim 9, wherein the compound of Formula XIIIa is prepared by
(1) contacting a compound of Formula XIa 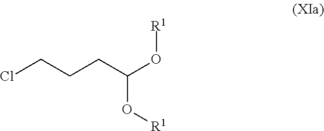
with a compound of Formula XII 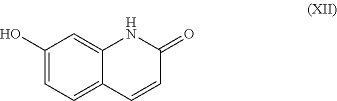 [SMILES: O=c1ccc2ccc(O)cc2[nH]1]
and a base in a first organic solvent to form a compound of Formula XIIIa.

19. The process of claim 18, further comprising a phase transfer reagent.

20. The process of claim 19, wherein the phase transfer reagent is selected from the group consisting of an ammonium salt, a phosphonium salt, or a combination thereof.

21. The process of claim 20, wherein the phase transfer reagent is an ammonium salt selected from the group consisting of tetra-n-butylammonium bromide (TBAB), tetra-n-butylammonium iodide, tetra-n-butylammonium bisulfate, tetra-n-butylammonium cyanate, tetra-n-butylammonium methoxide, tetra-n-butylammonium nitrate, and tetra-n-methyl ammonium bromide, or a combination thereof.

22. The process of claim 21, wherein the phase transfer reagent is TBAB.

23. The process of claim 18, wherein the first organic solvent is selected from the group consisting of dimethyl sulfoxide (DMSO), dimethylacetamide (DMAc), dichloromethane, and acetone.

24. The process of claim 23, wherein the first organic solvent is DMSO or DMAc.

25. The process of claim 18, wherein the base is selected from the group consisting of Li₂CO₃, K₂CO₃, Cs₂CO₃, Na₂CO₃, NaHCO₃, and KHCO₃, or a combination thereof.

26. The process of claim 25, wherein the base is K₂CO₃ or Na₂CO₃.

27. The process of claim 18, wherein the compound of Formula XIa is represented by a compound of Formula XI

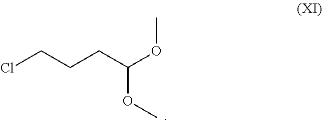

28.-44. (canceled)

45. A process for purifying brexpiprazole comprising:
(a) contacting brexpiprazole with a protic acid in an aqueous solution to form a brexpiprazole acid salt of Formula XVI 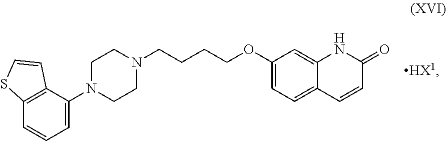
wherein X¹ is the anion of a protic acid;
(b) contacting the brexpiprazole acid salt with activated carbon;
(c) filtering the brexpiprazole acid salt and the activated carbon through a chemically inert filtration bed to produce a purified brexpiprazole acid salt;
(d) contacting the purified brexpiprazole acid salt with a base in a second aqueous solution to form purified brexpiprazole.

46. The process of claim 45, wherein step (a) further comprises
(a-iii) separating the brexpiprazole acid salt to form an isolated brexpiprazole acid salt,
wherein step (a-iii) is performed after contacting brexpiprazole with the protic acid in the aqueous solution.

47. The process of claim 46, wherein the aqueous solution in step (a-iii) further comprises
(a-i) heating the aqueous solution to about 50-70° C.;
(a-ii) cooling the aqueous solution to about 10-40° C.,
wherein step (a-i)-(a-ii) are performed before said separating step (a-iii).

48. The process of claim 45, wherein the aqueous solution of step (a) further comprises isopropyl alcohol.

49. The process of claim 45, wherein the protic acid is selected from the group consisting of HCl, HBr, HI, H₂SO₄, H₃PO₄, acetic acid, HNO₃, H₂SO₃, tosylic acid (TsOH), and methansulfonic acid.

50. The process of claim 49, wherein the protic acid is HCl.

51. The process of claim 45, wherein the anion of the protic acid (X¹) is selected from the group consisting of chloride, bromide, iodide, sulfonate, tosylate, mesylate, nitrate and acetate, or combinations thereof.

52. The process of claim 51, wherein the anion of the protic acid (X¹) is chloride.

53. The process of claim 45, wherein the protic acid is added in sufficient amount to adjust the pH of the aqueous solution to <3.

54. The process of claim 45, wherein the second aqueous solution of step (d) further comprises ethanol.

55. The process of claim 45, wherein the base is a metal hydroxide.

56. The process of claim 55, wherein the base is NaOH.

57. The process of claim 45, wherein step (d) further comprises
(d-iii) separating the brexpiprazole acid salt to form isolated purified brexpiprazole,
wherein step (d-iii) is performed after contacting the brexpiprazole acid salt with the second base in the second aqueous solution.

58. The process of claim 57, wherein step (d-iii) further comprises
(d-i) heating the second aqueous solution to about 60-90° C.;
(d-ii) cooling the second aqueous solution to about 10-40° C.,
wherein steps (d-i)-(d-ii) are performed before said separating step (d-iii).

59. (canceled)

60. A process for preparing brexpiprazole 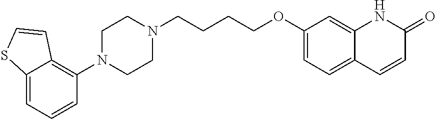 [SMILES: O=c1ccc2ccc(OCCCCN3CCN(c4cccc5sccc45)CC3)cc2[nH]1]
comprising:
a) converting the compound of Formula VI 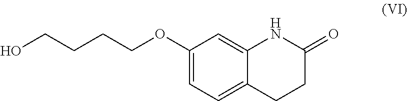 [SMILES: O=C1CCc2ccc(OCCCCO)cc2N1]
to a compound of Formula VII 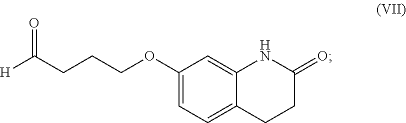 [SMILES: O=CCCCOc1ccc2c(c1)NC(=O)CC2]
b) contacting the compound of Formula VII with a compound of Formula II 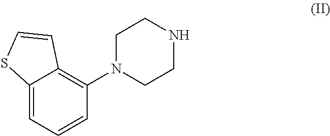 [SMILES: c1cc(N2CCNCC2)c2ccsc2c1]
to form a compound of Formula V 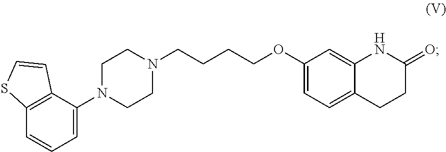 [SMILES: O=C1CCc2ccc(OCCCCN3CCN(c4cccc5sccc45)CC3)cc2N1]
and
c) converting the compound of Formula V to brexpiprazole.

61.-86. (canceled)

87. A pharmaceutical composition comprising brexpiprazole prepared according to the process of claim 1 and a pharmaceutically acceptable excipient.
